(19) United States
(12) Patent Application Publication
(10) Pub. No.: US 2014/0238406 A1
(43) Pub. Date: Aug. 28, 2014

(54) BREATHING TUBE HOLDER

(71) Applicants: Michael Kevin Borre, Glendale, CA (US); James Patrick Borre, Estero, FL (US)

(72) Inventors: Michael Kevin Borre, Glendale, CA (US); James Patrick Borre, Estero, FL (US)

(21) Appl. No.: 13/774,878

(22) Filed: Feb. 22, 2013

(51) Int. Cl. A61M 16/04

(52) U.S. Cl. CPC A61M 16/0497
USPC 128/207.17

(57) ABSTRACT

A faceplate assembly that includes a faceplate frame (14) that mounts on a patient's face and a bite block (30) that holds an endotracheal tube (22) that extends into a patient's trachea to assist breathing. The tube is easily mounted on the bite block, and the tube can be shifted sidewardly to reduce discomfort to the lips of the patient. The bite block has a passage (42) through which the tube extends, the passage being spaced (D) sideward from the faceplate centerline (60). The faceplate can be removed from the patient, turned 180° about the centerline, and reinstalled using adhesive tape, to shift the bite block to the opposite side of the patient's mouth.

### CROSS-REFERENCE

Applicant claims priority from U.S. provisional patent application Ser. No. 61/603,918 filed Feb. 27, 2012.

### BACKGROUND OF THE INVENTION

Some patients with respiratory ailments who need breathing assistance, require that an endotracheal tube, or breathing tube, extend into their trachea to carry air to the lungs, as from a controlled air pump. The tube extends from outside the patient, through the mouth, and into the trachea. The patient wears a facemask assembly at a predetermined position on the face. After awhile, the patient may experience discomfort due to the same spot on his/her lips lying clamped against the tube. The discomfort can be stopped by occasionally moving the tube to one side and then to the other side, of the mouth so different locations of the lips press against the tube. The time and effort required to shift the tube should be as little as possible.

### SUMMARY OF THE INVENTION

In accordance with one embodiment of the invention, a faceplate assembly is provided that includes a faceplate frame that mounts over a patient's mouth, and a bite block that mounts on the faceplate frame and that controls the position and orientation of the breathing tube in its passage through the mouth so as to pass into the patient's trachea. The faceplate frame has a centerline that extends horizontally and in a forward-rearward direction through the middle of the patient's mouth. The bite block guides the breathing tube to position it at one side of the centerline, so the tube is clamped between locations of the patient's lips that lie to one side of the centerline. To avoid pressure sores and provide relief from discomfort at the lip clamping locations, the tube is detached from the bite block, the faceplate assembly is turned 180°, and the tube is reattached to the bite block. This results in the tube being clamped between locations of the patient's lips that lie on the opposite side of the centerline. By occasionally moving the tube from one side of the mouth to the other side, applicant can avoid pressure sores on the patient's lips.

The breathing tube is mounted on the bite block by inserting it sideward into an open side of a tube guide of the bite block. Then an adhesive tape is wrapped around the tube and around the front of the bite block to fix them together. The adhesive tape can be easily removed, the faceplate frame turned 180°, and another piece of tape applied.

The novel features of the invention are set forth with particularly in the appended claims. The invention will be best understood from the following description when read in conjunction with the accompanying drawings.

### BRIEF DESCRIPTION OF THE DRAWINGS

FIG. 1 is a front and left side isometric view of a faceplate assembly with an endotracheal tube mounted thereon and with the tube extending forward and to the right of the faceplate centerline.

FIG. 2 is a front elevation view of the faceplate assembly and tube of 1.

FIG. 3 is a front elevation view similar to that of FIG. 2, but with the faceplate assembly having been turned 180< about the faceplate frame centerline from the orientation of FIG. 2.

FIG. 4 is a plan view of the apparatus of FIG. 2 in the orientation of FIG. 2.

FIG. 5 is a rear isometric view of the faceplate assembly of FIG. 1, without the tube.

FIG. 6 is sectional view taken on line 6-6 of FIG. 5.

FIG. 7 is a sectional view taken on line 7-7 of FIG. 4.

### DESCRIPTION OF THE PREFERRED EMBODIMENTS

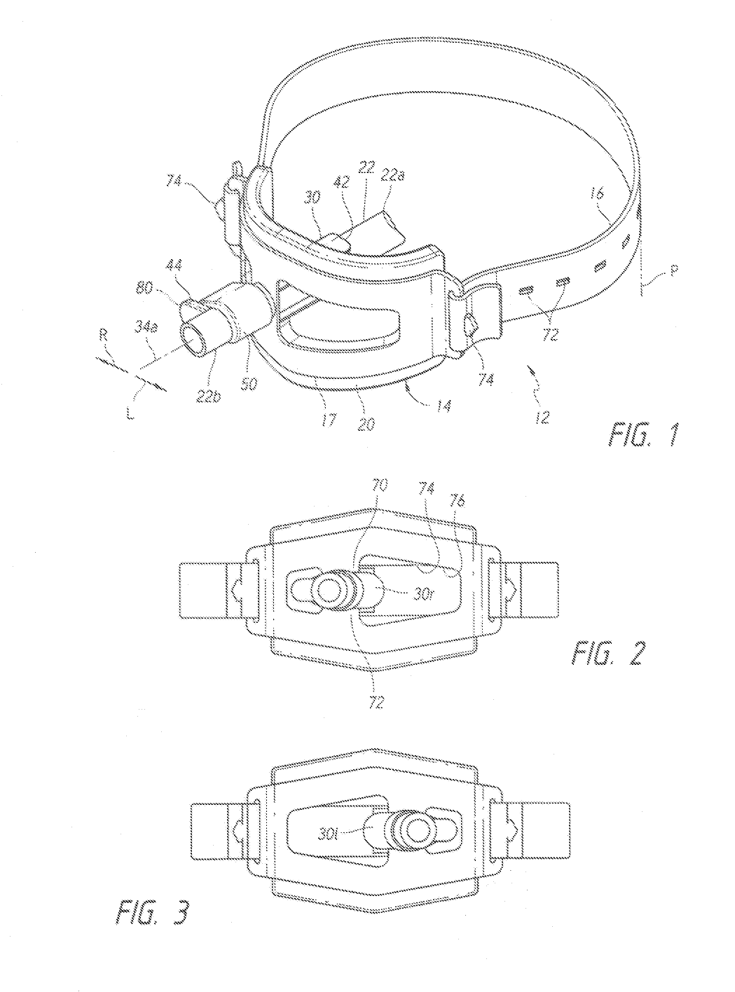

FIG. 1 shows a faceplate assembly 12 mounted on a patient P, and including a faceplate frame 14 mounted over the patient's face. An elastic band 16 holds the frame against the mouth region of the patient's face (the mouth region includes areas above and below the mouth). The faceplate frame includes a mask 17 of rigid material and a resilient foam backing 20 that actually contacts the patient's face. The figure also shows an endotracheal tube, or breathing tube 22 installed on the faceplate frame. The tube has a rear end 22A that extends to the patient's trachea and has an opposite front end 22B that extends to a source that repeatedly pumps air or other gas to the patient for breathing.

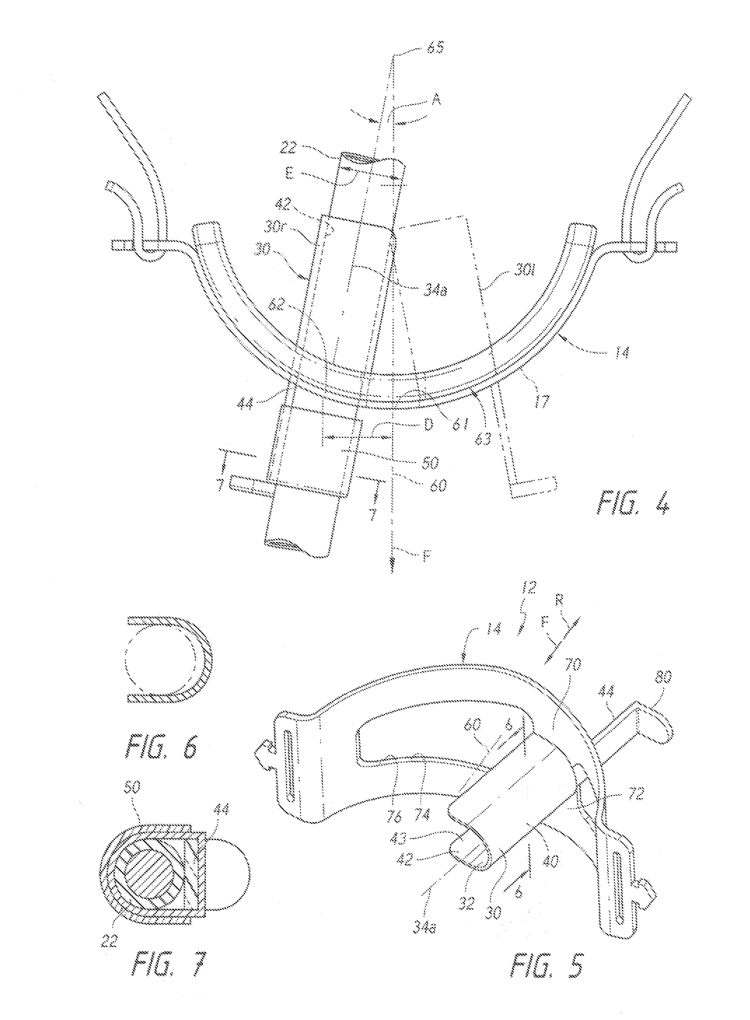

The faceplate assembly 12 (FIG. 5) includes a bite block 30 that has a passage 42 with a passage axis 34a that the breathing tube extends along. The bite block 30 has a rear tube guide part 40 that lies rearward of the middle of the faceplate frame 14 and that has an open side 43. The tube can be moved sidewardly through the open side to lie within the bite block passage. The bite block also has a front guide part 44 that has a flat part and a flange 80 and that extends forward of the faceplate frame. The breathing tube is inserted through the open side 43 of the bite block into the passage. Then adhesive tape is used that is wrapped about the front guide part 44 and around the breathing tube 22 to fix the tube in place. A strip of tape with Velcro fasteners on the ends, instead of adhesive, can be used instead. A length of adhesive tape is preferred. FIG. 4 shows the breathing tube fully installed on the bite block 30, with adhesive tape 50 wrapped about the bite block and the tube. FIG. 7 shows the adhesive tape 50 wrapped about the front guide part 44 of the bite block and around the tube 22.

FIG. 4 shows that the faceplate frame 14 has a centerline 60 that is coincident with a centerline that extends horizontally and through the middle of the patient's head and the middle of the patient's face and month. However, the bite block at 30R holds the breathing tube 22 so it extends along the bite block passage 42. In FIG. 1, the tube has been shifted to the right R and is also angled by an angle A (FIG. 4) from the centerline so the tube rear part easily reaches the patient's trachea. The tube is held so that the location 62 where the axis 34a of the bite block passage 32 crosses the faceplate frame, is spaced a distance D from a centerpoint 61 on the centerline 60. The centerpoint 61 lies at the middle of the faceplate frame where it contacts the patient's face, and along the centerline 60. The distance D is preferably at least one-half the diameter E of the breathing tube and at least five millimeters. As a result, the lips of the patient clamp against the bite block at 62 which is spaced from the centerline.

To relieve discomfort to the patient's lips, the breathing tube 22 is removed from the bite block. This is done by unwinding the adhesive tape 50 from the front part 44 of the bite block and sliding the breathing tube sidewardly out of the bite block. Then, the faceplate frame 14 is turned 180° about the centerline 60, so the bite block lies in the position shown at 30L in FIG. 4. In position 30L, the patient's lips clamp against the bite block at faceplate location 63. Then the tube is reinstalled in the bite block and taped in place.

As shown in FIG. 2, the faceplate has a pair of projections 70, 72 that project respectively downward and upward from the walls of a slot 74 in the faceplate frame. The bite block 30 is fixed between and mounted on the projections. The projection leave an access opening 76 in the faceplate frame through which instruments can be inserted as for dental hygiene, while the faceplate assembly is mounted on the patient. The maximum height of the opening 76 is greater than the distance between the projections 70, 72.

The faceplate assembly 12 of FIG. 1 is initially mounted on the head of a patient by pulling the elastic band 16 to tighten the band, and then inserting prongs 74 of the faceplate assembly through selected holes 72 of the band. After the breathing tube 22 has been installed on the bite block, the tape 50 is wrapped about the front end of the front guide part 44, immediately behind a flange 80.

In a faceplate assembly that applicant constructed, the faceplate frame 14 and bite block 30 were each formed of rigid plastic. The breathing tube 22 was formed of an elastomeric material and had an outside diameter of 13 mm. The passage axis of the bite block was spaced a distance D of 13 mm from the centerline 60 at the front of the faceplate frame 17. The axis 34a of the bite block should be angled by an angle A (FIG. 4) of 5° to 20° about a point 65 to properly route the breathing tube at the trachea, and was actually angled by 10°.

Thus, the invention provides a faceplate assembly that includes a faceplate that mounts over a patient's mouth, and a bite block that the patient's lips clamp onto and which the patient's teeth usually lie against. The breathing tube extends through a passage of the bite block and is attached to the faceplate assembly by a length of tape that wraps around the bite block and the tube. The faceplate frame has a centerline, and the bite block passage extends along an axis that is laterally horizontally spaced from the centerline at the center of the faceplate frame. As a result, the faceplate assembly can be turned upside down, or 180°, from one position wherein the tube extends to the right of the centerline at the faceplate frame, to a position wherein the tube lies to the left of the centerline. The shifting of the bite block and therefore of the tube, allows different side of the patient's lips to clamp on the tube at different times.

Although particular embodiments of the invention have been described and illustrated herein, it is recognized that modifications and variations may readily occur to those skilled in the art, and consequently, it is intended that the claims be interpreted to cover such modifications and equivalents.

What is claimed is:

1. Apparatus for mounting an endotracheal tube on a patient, comprising:
a faceplate assembly (12) which includes a faceplate frame (14) that mounts on a patient's face and that has a centerline (60) that is horizontal and that extends through the middle of the patient's face and the middle of the back of his head;
said faceplate frame having a centerpoint (61) that lies on said centerline where the middle of the faceplate frame lies at the middle of the patient's mouth;
said faceplate assembly including a bite block (30) that is mounted on said frame and that has a passage (42) with a horizontal passage axis (34a), said passage being constructed to hold said endotracheal tube in extension along said passage axis;
an endotracheal tube (22) that extends through said passage;
said faceplate being installable on the patient in a first orientation wherein said endotracheal tube (30L) is spaced leftward from said centerpoint, and said faceplate being installable in an upside-down orientation on the patient wherein said endotracheal tube (30R) lies rightward of said centerpoint.

2. The tube holder described in claim 1 wherein:
said bite block surrounds a first side of said passage axis but not a second side, and said bite block has a horizontal slot (43) at said second side that passes said endotracheal tube out of and into said passage.

3. The tube holder described in claim 1 including:
a length of adhesive tape (50) that is wrapped around said bite block and said tube to fix said tube in place.

4. The tube holder described in claim 1 wherein:
said bite block includes a front guide part (44) which has a U cross-section with horizontal top and bottom walls, and said bite block has a front portion (44) that extends partially about said passage axis, and said tape is wrapped about said front portion of said bite block.

5. The tube holder described in claim 1 wherein:
said passage axis (34a) is angled (A) from said centerline about a point lying a plurality of centimeters rearward of said centerpoint.

6. Apparatus for mounting an endotracheal tube on a patient, comprising:
a faceplate which includes a faceplate frame (14) that mounts on a patient's face at his mouth region, and a bite block (30) fixed to said faceplate frame and having walls forming a tube guide with a front guide portion (44) that extends forward of said faceplate and a rear guide portion (44) that extends rearward of said faceplate, and said tube extending along end against walls of said front and rear guide portions in extension along a tube axis (34a) through said faceplate;
a length of adhesive tape (50) that extends around said front guide portion (44) and around a location on said tube that lies against said front guide portion to fix them together.

7. The apparatus described in claim 6 wherein:
said faceplate frame has a faceplate centerline (60) that is horizontal and aligned with a centerline of the patient's head when the faceplate frame is mounted thereon, and said faceplate frame has a centerpoint (61) lying on said centerline at a front of said faceplate frame;
said bite block lies at one side of said centerpoint;
in a first orientation of said faceplate said tube axis lies at least five millimeters to one side of said centerpoint and in another orientation of said faceplate that is turned 180° from said first orientation about said centerline said tube axis lies at least five millimeters to a second side of said centerpoint.

8. The apparatus described in claim 1 wherein:
said faceplate has a horizontal slot of a plurality of inches width that leaves top and bottom slot walls, and said faceplate has projections (72, 70) that project respectively from the bottom and top of said slot walls into the slot and hold said bite block, said projections located only at a first side of said centerline.

9. A method for using a faceplate apparatus to position an endotracheal tube by mounting the tube in a passage (42) of a bite block of the faceplate apparatus wherein the faceplate apparatus has a centerline (60) and has a centerpoint (61) that lies en said centerline and that lies at the center of the patient's month, comprising:
fixing said bite block so the bite block passage (42) is spaced rightward (30R) of said centerpoint, and installing said faceplate on the patient's mouth;
removing said faceplate apparatus from the patient's mouth, turning the faceplate apparatus 180° about said centerline, and again mounting the tube in said passage of the bite block and mounting the faceplate apparatus in the patient's mouth so the bite block passage is spaced leftward (30L) of said centerpoint.

10. The method described in claim 6 wherein:
said bite block has front and rear portions (44, 40) lying respectively forward and rearward of said face mask;
said steps of mounting the tube includes placing the tube in a passage (42) of said bite block rear portion, and placing the tube against said bite block front portion (44) and wrapping a length of adhesive tape (50) around said tube and bite block front portion.
